(19) United States
(12) Patent Application Publication
(10) Pub. No.: US 2007/0196303 A1
(43) Pub. Date: Aug. 23, 2007

(54) STABLE BUFFERED ALUMINUM COMPOSITIONS HAVING HIGH HPLC BANDS III AND IV CONTAINING CALCIUM/STRONTIUM

(75) Inventors: Zijun Li, Westfield, NJ (US); Mark Rerek, Scotch Plains, NJ (US)

Correspondence Address: ARTHUR J. PLANTAMURA;GENERAL CHEMICAL PERFORMANCE PRODUCTS LLC., 90 EAST HALSEY ROAD, PARSIPPANY, NJ, 07054, US

(73) Assignee: REHEIS, INC., Berkeley Heights, NJ (US)

(21) Appl. No.: 11/307,689

(22) Filed: Feb. 17, 2006

(51) Int. Cl. A61K 8/26

(52) U.S. Cl. 424/68

(57) ABSTRACT

A novel aluminum antiperspirant active composition of enhanced efficacy is provided. The composition is activated by both heat and acid and characterized by having high HPLC Band III and high HPLC Band IV, prepared by the addition of an acid such as AlCl₃ to (1) the aluminum calcium and/or strontium betaine solutions, where the calcium and/or strontium is derived from a soluble salt such as calcium and/or strontium chloride or nitrate and (2) the aluminum calcium and/or strontium glycine and/or betaine solutions, where calcium and/or strontium is from strongly alkaline calcium/strontium oxide or hydroxide. The novel aluminum compositions derived according to the invention have HPLC Band I of less than 2%; a Band III of at least 20%; a Band III/II ratio of at least about 0.7; and a Band IV of at least 20%.

This invention relates to novel aluminum antiperspirant compositions of enhanced efficacy that are activated by both heat and acid and have a high HPLC Band III as well as a high HPLC Band IV and to the processes for the preparation of the compositions. The processes include the addition of an acid such as AlCl₃ to either the aluminum calcium betaine solutions, or to the aluminum strontium betaine solutions, or to the aluminum calcium/strontium glycine solutions where calcium and/or strontium is derived from strongly alkaline oxides or hydroxides of either calcium and/or strontium. The invention relates as well to the use of these compositions in consumer acceptable antiperspirant vehicles such as aerosols, gels, roll-on, sticks and soft solids.

### BACKGROUND OF THE INVENTION

Enhanced efficacy aluminum antiperspirant salts are well known and generally related to the aluminum species that are activated by either heat as indicated by having high SEC (size exclusion chromatography)-HPLC (high performance liquid chromatography) Band III or by acid as having high Band IV content at lower Al/Cl ratio. These compositions are typically different from conventional antiperspirant salts that have low Band III, with Band III/II area ratio of less than 0.1 or 0.2, as well as low Band IV of less than 20%. In the evaluation of “Bands” and “peaks”, as applied in the characterization for antiperspirants, typically Bands I, II, III and IV of one system correspond to peaks 1, 2, 3, 4 and 5 of the other system, i.e., with Band I corresponding to peak 1+peak 2, Band II corresponding to peak 3, Band III corresponding to peak 4 and Band IV corresponding to peak 5. Enhanced efficacy aluminum antiperspirant salts have an HPLC Band III/II area ratio of 0.5 or higher with at least 70% and preferably at least 80% of aluminum contained in Bands II and III. Thus, the enhanced salts typically have a Band III content of at least 20%, and more likely at least 30%, of the total aluminum contained in all the peaks as measured by peak area. In contrast, conventional non-enhanced antiperspirant salts have Band III content in the range of about 10% and Band III/II area ratio from 0.1 to 0.2 or less. Typically, the enhanced aluminum salts activated by heat, which is characterized by the presence of high HPLC Band III or high Band III/II ratio, are prepared by the dilution and heat treatment of 50% ACH (aluminum chlorohydrate) solution and drying the solutions to powders because of the rapid reverting back of Band III to Band II when such are in aqueous solutions, particularly at concentrations of greater than 20%.

Prior art patents describing the enhanced aluminum salts include British Patent GB 2,048,229; U.S. Pat. No. 4,359,456; U.S. Pat. No. 4,775,528; U.S. Pat. No. 4,818,512; U.S. Pat. No. 4,859,446; U.S. Pat. No. 4,871,525; U.S. Pat. No. 4,900,534; U.S. Pat. No. 4,944,933; U.S. Pat. No. 5,356,609; U.S. Pat. No. 5,358,694; U.S. Pat. No.5,595,729 and U.S. Pat. No. 5,626,827.

U.S. Pat. No. 6,042,816 describes a method of stabilizing aqueous solutions of enhanced efficacy aluminum and aluminum-zirconium antiperspirant salts, containing an effective amount of a soluble calcium salt and an effective amount of a water soluble amino acid, such as glycine, against rapid degradation of HPLC peak 4 (Band III) to peak 3 (Band II) ratio of the salts. However, high concentration aluminum salt solutions with high peak 4/3 (aka Band III/II) ratio, stabilized by calcium chloride dehydrate in the presence of glycine are not stable, the solutions gel quickly at room temperature (RT). Further, the aluminum-calcium-glycine solutions as disclosed in U.S. Pat. No. 6,042,816 have Band IV of less than 20%, typically less than 10%.

U.S. Publication No. 2005/0265939 discloses aluminum and aluminum-zirconium compositions of enhanced efficacy having high pH prepared by the reaction of aluminum chloride aqueous solutions with insoluble, strongly alkaline calcium or strontium salts, followed by the addition of aluminum powder at high temperatures and although the resultant solutions are disclosed as stable and have high HPLC Band III, the Band IV peak areas of the aluminum-calcium solutions are low, usually less than 10%.

U.S. Publication No. 2004/0091436 discloses antiperspirant actives of aluminum and aluminum-zirconium of enhanced efficacy containing strontium and an amino acid. The strontium salts both activate and stabilize HPLC Band III in the presence of glycine at high aluminum and aluminum-zirconium solution concentrations. Here again, the Band IV areas of the aluminum-strontium solutions are low, i.e., considerably less than 20%.

Aluminum antiperspirant salts of enhanced efficacy can also be prepared through acid activation at lower Al/Cl ratio, such as ratios of less than 1.5 as described, for example, in U.S. Pat. Nos. 5,718,876 and 6,024,945, where the basic aluminum chloride solutions with 28% to 42% solid concentration have at least 50% HPLC Band II and less than 20% Band III. Further, U.S. Pat. No. 6,902,724 describes a basic aluminum chloride (BAC) composition having a Band I relative area value of less than 5%, a Band II of 20% to 60%, a Band III of 10% to 35% and a Band IV of 15% to 50% at Al/Cl ratio of 1.2 to 1.5. The BAC solution is prepared by reacting aluminum powder with aluminum chloride aqueous solution at a temperature greater than 85° C. In the examples disclosed in that patent, e.g., Examples 1, 6, 7 and 8, the freshly prepared BAC solutions always have Band III of less than 20% and in any event no BAC solution in that reference appears as having a Band III area greater than 25%. Moreover, the HPLC figure in that patent focuses on a BAC solution having high Bands II and IV.

In addition to amino acids, such as glycine, which has been used widely as buffers in aluminum-zirconium antiperspirants, other buffers such as betaine have also been used in antiperspirants. Betaine, also identified as trimethylglycine, and its derivatives, including the monohydrate and hydrochloride solids, is not regarded as an amino acid. Trimethylglycine (betaine) has a quarternary nitrogen group that cannot act as a hydrogen ion donor or acceptor in place of an amino group. The normal form of the compound is as an internal salt, or zwiterion, of the composition (CH₃)₃N⁺CH₂COO⁻. As such, it is regarded as having a very different chemistry from glycine. Examples of recent publications on betaine are referenced below.

WO 2004/026295A2 relates to the use of zwitterionic compounds and their derivatives as protecting agents of skin, hair and nails. The zwitterionic compounds include betaines. Additional betaine disclosures include WO 2004/089325 A1, EP 1005853 B1, U.S. Publication No. 2004/0109833 A1 and U.S. Publication No. 2004/0198998 A1.

U.S. Pat. No. 6,969,510 describes a glycine-free aluminum and/or zirconium betaine salt, where betaine is used as either the monohydrate or the hydrochloride derivative. While a number of different methods are disclosed in the prior art literature for making betaine containing antiperspirant salts, none of the known methods describes a process to activate basic aluminum chloride in the presence of betaine to yield a composition characterized by high Band III coupled with a high Band IV. That disclosure encompasses a method for the addition of betaine to previously activated aluminum chlorohydrol solutions followed by drying the solutions to powders.

Heretofore stable antiperspirant solutions at concentrations of greater than 20% containing aluminum-only actives of enhanced efficacy that are activated by both heat and acid with high HPLC Band III and high HPLC Band IV were not known.

Accordingly, based on an appraisal of the prior art, it is regarded as highly desirable to provide aluminum antiperspirant compositions of enhanced efficacy that are activated by both heat and acid and that have both a high HPLC Band III and a high Band IV. The aluminum solutions of the invention can be prepared at higher concentration and are relatively stable in aqueous solution compared to solutions solely prepared by heat activation. Additionally, the solutions of the invention can be dried to powders. Both the aluminum solutions and powders prepared according to the invention can be used in various finished formulations, such as aerosols, gels, roll-ons, sticks, soft solids and the like.

### SUMMARY OF THE INVENTION

According to the present invention, it has been discovered that novel aluminum compositions of enhanced antiperspirant efficacy that are activated by both heat and acid and are characterized by having high HPLC Band III and high HPLC Band IV, are prepared by a process, referred to for convenience as Process “A”, the addition of an acid such as AlCl₃ to the aluminum calcium or strontium or calcium strontium betaine solutions, where the calcium and/or strontium is derived from a soluble salt such as calcium and/or strontium chloride or nitrate. Alternatively, these novel aluminum calcium and/or strontium glycine and/or betaine solutions, may be prepared by an alternative process referred to as Process “B” wherein calcium and/or strontium is derived from strongly alkaline calcium and/or strontium oxide or hydroxide. The novel aluminum compositions derived according to the invention have HPLC Band I of less than 2%, preferably about 0%; a Band III of at least 20%, preferably about 30%; a Band III/II ratio of at least about 0.7, preferably about 0.9; and a Band IV of at least 20%, and preferably about 25%. The resulting novel aluminum compositions usually have 20% to 40% anhydrous solids preferably 25% to 35%. The compositions have Al/Cl ratio of below 1.5. The activated buffered aluminum calcium/strontium salt solutions can be used as is or be dried to powders for use in aerosol, soft solid, cream, roll-on, gel, stick formulations, as appropriate.

Accordingly, it is an object of the present invention to provide novel stable high concentration basic aluminum halide antiperspirant solution actives activated by both heat and acid which can be produced economically. Optionally, these solutions can be dried to solids more efficiently than conventionally heat activated aluminum halides.

It is another object of the present invention to provide basic aluminum halide antiperspirants of this kind that have substantially enhanced efficacy and to provide methods of forming such materials without the need for manufacturing steps previously thought to be necessary, such as for example, the need to heat diluted solutions of already manufactured basic aluminum halides at high temperatures and/or the requirement of use of pressure conditions, and to further activate the high Band III aluminum solution by acid.

It is yet another object of the present invention to provide antiperspirant compositions having greater antiperspirancy and skin friendliness.

It is another object of this invention to provide antiperspirant compositions that contain aluminum only actives of greater efficacy that can be used in aerosol formulation with much lower corrosion potential than conventional acid activated actives.

It is another object of this invention to provide a process for the preparation and stabilization of concentrated BAC solutions with high HPLC Band III/II ratio and with a high Band IV area that possess excellent physical and chemical stability and that can be incorporated in enhanced efficacy gel and emulsion antiperspirant products.

### BRIEF DESCRIPTION OF THE DRAWINGS

FIG. 1 is the SEC-HPLC of an aluminum-betaine-calcium salt solution with an Al/Cl ratio of 1.2 prepared according to the process of the invention.

FIG. 2 is the ²⁷Al NMR of an aluminum-calcium-glycine salt solution where the calcium is derived from calcium oxide prepared according to the invention.

### DETAILED DESCRIPTION OF THE INVENTION

The present invention is directed to a novel betaine containing aluminum calcium/strontium and/or aluminum calcium/strontium glycine antiperspirant compositions of enhanced efficacy activated by both heat and acid which, when analyzed by SEC-HPLC at about 10% concentration (i.e., by 2% Al) in water, exhibit an HPLC Band I area of less than 2%, preferably about 0%; a Band III area of at least 20%, preferably 30% or more; a Band III/II ratio of at least 0.7, preferably 0.9 or more; a Band IV area of at least 20%, preferably 25% or more; and having at least 60% and preferably 70% or more aluminum species contained in Bands III and Band IV.

The basic aluminum salts of these compositions have the formula:
Al₂(OH)₆₋ₐXₐ
wherein X is Cl⁻, Br⁻, I⁻ or NO₃⁻, a is from about 1.4 to about 2, with basic aluminum chloride the most preferred. The acronym BAC, while used to designate basic aluminum chloride, is herein intended to include other halogen ions other than chloride and the nitrate ion NO₃⁻ as well. Also, the abbreviation Ca/Sr is intended to include Ca or Sr or a blended mixture of Ca and Sr. The preferred ratio of aluminum to chloride of the BAC solution depends on the type of calcium and/or strontium compound used in the activation, i.e., a water soluble salt such as calcium and/or strontium chloride or insoluble, highly alkaline base such as calcium oxide or strontium hydroxide or blends thereof.

When betaine is present in the double activated Al—Ca/Sr-betaine solution that has both high HPLC Bands III and IV, the resultant aluminum solution is stable irrespective of the type of calcium/strontium compound used. As used herein the term “double activated” means that both heat and acid induce the described reaction. The preferred Al/Cl ratio for the double activated Al—Ca/Sr-betaine solution is about 1.2 to about 1.3 when soluble calcium/strontium salt such as calcium chloride dihydrate is used and is about 1 to about 1.2, where the calcium/strontium is from an insoluble and strongly alkaline base such as calcium oxide/or strontium hydroxide.

When glycine is used to make the novel Al—Ca/Sr-glycine solution that has both high HPLC Band III and Band IV contents and where the Ca/Sr is from a soluble compound such as calcium/strontium chloride, the solution quickly became hazy upon the addition of AlCl₃, and creamy white after standing overnight. Not wishing to be bound by any particular theory, it is believed that the high chloride content is responsible for the formation of the insoluble white precipitates. However, when the Ca/Sr used is derived from an insoluble strongly alkaline compound, such as calcium oxide, a clear Al—Ca/Sr-glycine solution is formed upon the addition of the AlCl₃. The stability of the solution depends on the amounts of glycine and calcium/strontium, as well as on the Al/Cl ratio of the BAC solution. A small amount of white precipitate is formed on aging at room temperature at higher glycine content or at lower Al/Cl ratio or under both conditions. The solution is stable at lower amounts of glycine or higher Al/Cl ratio or under both conditions. The preferred Al/Cl ratio for the Al—Ca/Sr-glycine solution, where the Ca/Sr is from an insoluble strongly alkaline compound, is between 1 to 1.2.

According to the present invention it has been discovered that the degree of the heat activation of the basic aluminum chloride solutions, in order to achieve high Band III, is largely dependent on the basicity of the solutions. The basicity of the aluminum composition is defined as the ratio of moles of hydroxide to three times the moles of the aluminum of the composition. The higher the basicity of the solution, the higher the Al/Cl ratio, the easier the activation becomes, and the higher HPLC Band III area that can be obtained. Generally, at Al/Cl ratio below 1.5, it is relatively difficult to heat activate an aluminum solution, since the aluminum species in the solution are already depolymerized. A soluble calcium/strontium salt usually does not change the basicity of the aluminum solution in the presence of a buffer such as glycine or betaine. The presence of a soluble calcium/strontium salt in the BAC solution at lower Al/Cl ratio appears not to activate Band III at higher Al concentration even in the presence of glycine or betaine. The introduction of an insoluble strongly alkaline calcium/strontium compound, however, increases the basicity of the BAC solution significantly. For example, an aluminum dichlorohydrate calcium solution made from calcium oxide with 8.87% Al, 9.45% Cl and 1.85% Ca has a calculated basicity of 82.4%, which is similar to the basicity of the aluminum chlorohydrate (ACH) solution, compared to 72.9% calculated basicity of the similar aluminum dichlorohydrate solution in the absence of calcium oxide or in the presence of a soluble calcium salt such as calcium chloride dihydrate, whereas the BAC solution at Al/Cl ratio of 1.5 has a calculated basicity of 77.8%. The former solution is easily activated through the heat treatment of the solution in the presence of either glycine or betaine. For example at 6% betaine the solution has about 63% Band III, while it is very difficult to heat activate the aluminum dichlorohydrate solution in the presence of calcium chloride dihydrate.

When using the heat activation process of the present invention (referred to as Process A), the BAC solutions are mixed with a water soluble calcium and/or strontium salt, preferably calcium and/or strontium chloride, and betaine then heated from about 40° C. to about reflux until an HPLC Band III/II ratio of at least 0.7 is achieved. The preferred Al/Cl ratio of the BAC solution is from 1.6 to 1.9, with a preferred ratio being 1.7 to 1.8. When ACH solution (Al/Cl=1.91-2.1) is used, a higher HPLC Band I is formed and the activated species are not stable at higher concentrations.

In a modified method of the invention (referred to as Process B) AlCl₃ (or HCl) is reacted with a strongly alkaline base such as CaO or Sr(OH)₂ or a blend thereof, for example, and aluminum powder is added and reacted at high temperature of greater than 80° C. and then filtering the solution. The preferred Al/Cl ratio for the BAC-Ca/Sr solution is from about 1.2 to about 1.5. This method permits the utilization of an amino acid buffering agent such as glycine as well as betaine or a mixture thereof. The buffer such as glycine or betaine is added to the filtered solution and heated to obtain the desired activation. An acid is then added to the high HPLC Band III Al—Ca/Sr-betaine/glycine solution to further activate the Band III solution in order to obtain the final high efficacy solution having both high Band III and IV. The acids include AlX₃, wherein X is Cl⁻, Br⁻, I⁻ or NO₃⁻, with chloride the most preferred, and strong inorganic acids such as HCl and nitric acid. Aluminum dichlorohydrate can also be used as the acid. The novel aluminum solutions have an Al/Cl ratio of less than 1.5, and the preferred ratio depends on the type of Ca/Sr compound used in the preparation. When the aluminum solution prepared according to the invention is dried, the double activated aluminum active powder can be used in aerosol formulations, which prohibit the use of zirconium.

The double activated (by both heat and acid) aluminum antiperspirant solution of the present invention comprises about 20% to about 40% of basic aluminum chlorohydrate salt, preferably about 25% to about 35%. When betaine is used as the buffer, the composition comprises about 1% to about 8% by weight betaine, preferably about 3% to about 6% by weight and about 0.2% to about 8% by weight Ca and/or Sr, and preferably about 0.5% to about 4% by weight Ca and/or Sr. When an amino acid such as glycine is used as the buffer, the composition comprises about 1% to about 6% by weight of the amino acid, e.g., glycine, preferably about 2% to about 4%; and about 0.2% to about 8% by weight Ca and/or Sr, and preferably about 0.5% to about 4% by weight Ca and/or Sr. Typical water soluble calcium salts include calcium chloride, calcium nitrate, calcium bromide, calcium citrate, calcium formate, calcium acetate, calcium ascorbate, calcium sulfate, calcium lactate and mixtures thereof. Typical insoluble, alkaline calcium bases include calcium carbonate, calcium hydroxide, calcium oxide and mixtures thereof. Preferred water soluble strontium salts include strontium chloride, strontium nitrate, strontium bromide, strontium citrate, strontium formate, strontium acetate, strontium ascorbate, strontium lactate and mixtures thereof. Typical insoluble, alkaline strontium bases include strontium carbonate, strontium hydroxide, strontium oxide and mixtures thereof.

The amino acids, in addition to glycine, include alanine, lysine, ornithine, citrulline, hydroxyproline, cysteine, threonine, valine, serine, methionine, glutamic acid and the mixtures thereof. Glycine is the preferred amino acid. Betaine, also identified as trimethylglycine, and its derivatives including the monohydrate and hydrochloride solid derivatives, can be used in the present invention as buffers, as well as the stabilizing agents with respect to aluminum solution actives.

A polyhydric alcohol, such as propylene glycol, may be included in the aluminum composition, which has the effect of enhancing the stability of the corresponding solutions, especially with respect to inhibiting gelling.

The double activated aluminum solutions containing calcium/strontium salts and betaine and/or glycine can be dried to powders by any appropriate means, including freeze-drying and vacuum drying. Spray drying is the most preferred method. Furthermore, the solutions can be dried into different densities and different shapes, such as low and high density spherical particles to suit for the variety applications in the formulations like soft solids and low residue sticks.

The aluminum-calcium/strontium-betaine/glycine antiperspirant salts of the present invention are characterized by the following description:

SEC-HPLC

The degree of the polymerization of aluminum complexes is determined by Size Exclusion Chromatography (SEC) operated via a High Performance Liquid Chromatograph (HPLC) instrument. In this technique, the highest molecular weight Al species are eluted first and are designated as Band I, which is equivalent to peaks 1 and 2; Band II (equivalent to peak 3) and Band III (equivalent to peak 4) are intermediate molecular weight Al compounds. Band IV (equivalent to peak 5) is derived from the lowest molecular weight Al complexes, including monomers and dimers. Band V according to present invention is derived from calcium and/or strontium while Band VI is attributable to chloride. The relative area of one or more peaks is determined in order to characterize the distribution of polymeric species in the aluminum complexes are formed. All the aluminum species are eluted in Bands I, II, III and IV and the percentage of each peak is calculated accordingly without the inclusion of Band V or Band VI or Ca/Sr or chloride peaks, respectively. The aluminum antiperspirant salts containing betaine/or glycine and calcium/or strontium salts according to the present invention have SEC-HPLC Band III to Band II area ratio of at least 0.7, and most preferably at least 0.9. Band IV content should be at least about 20%. At least 60%, and preferably at least about 70%, of the total SEC-HPLC band content should be contained within Band III and Band IV. These enhanced salts have a Band III content of at least 20%, and preferably at least 30%, of the total aluminum contained in all the peaks as measured by peak area. No more than 2% aluminum species should be contained in Band I.

A trimethyl siloxane column (150×4.6 mm) from YMC. Inc., a Column Engineering Cl column (100×4.6 mm) and a Lichrosorb Silica column (250×4.6 mm) connected in series is used to obtain the SEC-HPLC Chromatograms. Each sample is dissolved in deionized water to form a 2% by weight Al solution. Each sample is filtered through a 0.45μ filter and chromatographed within 15 minutes using a 0.01N nitric acid solution as the mobile phase at a flow rate of 0.75 ml/minute.

Nuclear Magnetic Resonance Spectroscopy

²⁷Al Nuclear Magnetic Resonance (NMR) is utilized to identify the structures of different aluminum species in the double activated aluminum antiperspirant salts of enhanced efficacy. The antiperspirant salt in solution form is measured as is and the powder is dissolved in deuteriated water to form a 10% by weight solution just before the measurement. Data were collected using a Varian Inova 400 instrument at 104.2 MHz.

The invention will be further illustrated by the following Examples. In the Examples, parts are by weight unless otherwise specified.

### EXAMPLE 1

### Preparation of Aluminum-Calcium-Betaine Solution with Al/Cl Ratio of 1.2 According to Process (A)

4174 parts of Reach® 301 (an antiperspirant active) (comprising, 11.5% Al, 9.0% Cl and available from Reheis, Inc, Berkeley Heights, N.J.); 496 parts of calcium chloride dihydrate (available from Aldrich Chemicals, Inc.); 240 parts of betaine anhydrous (available from Arch Chemicals, Inc.); and 500 parts of water were mixed and refluxed for about 2 hours.

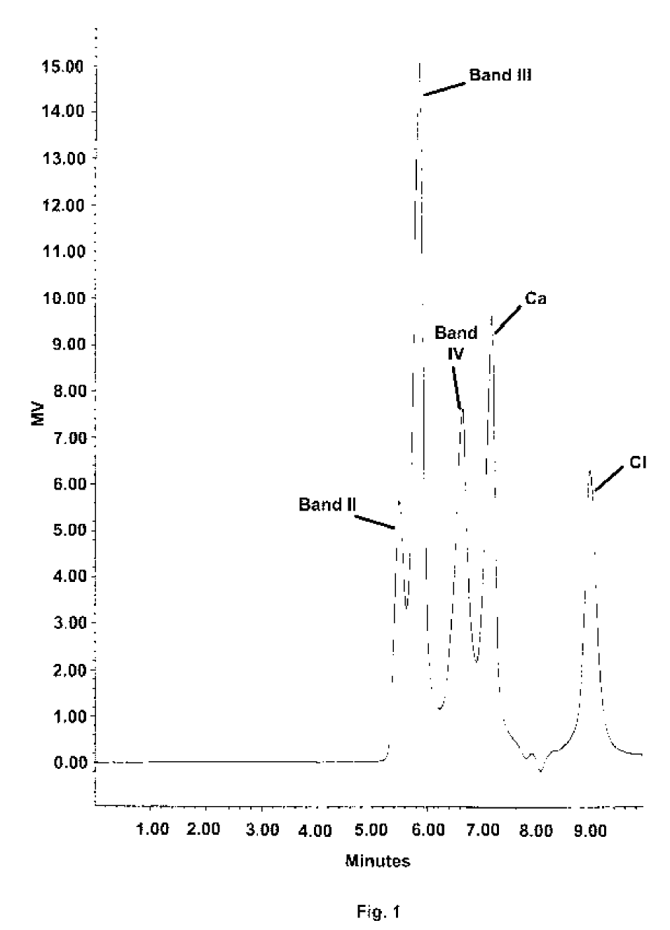

16 parts of AlCl₃ (32⁰Be) was gradually added to 100 parts of the above prepared solution i and the final Al—Ca-betaine solution has an Al/Cl ratio (excluding the Cl from calcium chloride dihydrate) of 1.2 and 30% anhydrous solids (A.S.). The resulting combined solution is clear and has a viscosity of less than 5 cps after two months. The HPLC of the solution is shown in FIG. 1. A similar Al—Ca-betaine solution having a Al/Cl ratio of 1.3 was also prepared. The HPLC data of the two solutions are summarized in Table I.

TABLE I

| | AI/CI Ratio 1.2 | | AI/CI Ratio 1.3 | |
|---|---|---|---|---|
| | % Band III | % Band IV | % Band III | % Band IV |
| fresh | 50.1 | 33.3 | 48.3 | 32.7 |
| 11 days | 43.5 | 36.9 | — | — |
| 2 weeks | 39.9 | 36.5 | — | — |
| 1 month | 35.5 | 39.4 | 39.5 | 30.4 |
| 2 months | 33.3 | 39.7 | — | — |

As can be seen from the data in Table I, both of the double activated Al—Ca-betaine solutions demonstrated high HPLC Bands III and IV. On aging the percentage of Band III content decreases and the percentage of Band IV increases, especially at lower Al/Cl ratios.

### EXAMPLE 2

### Preparation of Aluminum-Calcium/Strontium-Glycine Solution at Al/Cl Ratio of 1.3 and Comparison to the Corresponding Solutions Containing Betaine

Four activated solutions: (1) Al—Ca-glycine; (2) Al—Sr-glycine; (3) Al—Ca-betaine; and (4) Al—Sr-betaine were prepared using refluxed Reach® 301 (Al/Cl ratio 1.68), CaCl₂.2H₂O (available from Aldrich Chemical, Inc.)/or SrCl₂.6H₂O (available from NOAH Technologies Corporation) glycine/or betaine and small amount of water for a 2 hour reflux. The clear solutions have 2.5% Ca or 3.5% Sr, 2.84% glycine or 4.44% betaine, and about 29% A.S. Among the four solutions, the Al—Ca-glycine and Al—Sr-glycine solutions turned hazy and became creamy white on aging.

Small amount of AlCl₃ (32⁰Be) solutions were added to the above four freshly prepared solutions to make the double activated solutions with both high HPLC Bands III and IV at about 29% A.S. A hazy solution was formed immediately for Al—Ca-glycine, which turned creamy after overnight. The Al—Sr-glycine solution became hazy on aging. The Al—Ca/Sr-betaine solutions, by contrast, were clear after about two months.

TABLE II

| Experiment | % | | | | % Band | |
|---|---|---|---|---|---|---|
| Number | Ca | % Sr | % Glycine | % Betaine | III | % Band IV |
| 1 | 2.3 | — | 2.5 | — | 49.3 | 26.9 |
| 2 | — | 3.1 | 2.5 | — | 38.8 | 28.9 |
| 3 | — | 3.1 | — | 4.0 | 33.1 | 27.1 |
| 4 | 2.3 | — | — | 4.0 | 48.3 | 31.7 |

### EXAMPLE 3

### Preparation of Aluminum-Calcium/Strontium-Glycine/Betaine Solutions at Al/Cl Ratio of 1.25 through Direct Activation of Ca/Sr Chloride with BAC Solutions

Four solutions of Al—Ca-glycine, Al—Sr-glycine, Al—Ca-betaine, and Al—Sr-betaine were prepared through the reflux of BAC solutions (Al/Cl, 1.25), CaCl₂.2H₂O (available from Aldrich Chemical, Inc.)/or SrCl₂.6H₂O (available from NOAH Technologies Corporation), glycine/or betaine and small amount of water for 2 hours to have the similar concentrations as solutions ii in Example 2. The results are shown in Table III.

TABLE III

| Experiment | % | | | | % Band | |
|---|---|---|---|---|---|---|
| Number | Ca | % Sr | % Glycine | % Betaine | III | % Band IV |
| 5 | 2.5 | — | 3.0 | — | 21.0 | 35.4 |
| 6 | — | 3.4 | 3.5 | — | 18.7 | 39.8 |
| 7 | — | 3.4 | — | 4.5 | * | 39.1 |
| 8 | 2.5 | — | — | 4.5 | 25.5 | 46.6 |

* appears as a shoulder

White precipitates started forming for solution 5 when the solution was aged for two weeks at room temperature. It is surprising that the percentage of Band III for solution 8, i.e., Al—Ca-betaine solution increased to 33% after aging for two months.

### EXAMPLE 4

### Aluminum-Calcium-Glycine Solutions with Al/Cl Ratio of 1 and 1.1 with Different Amount of Glycine Prepared According to Process (B)

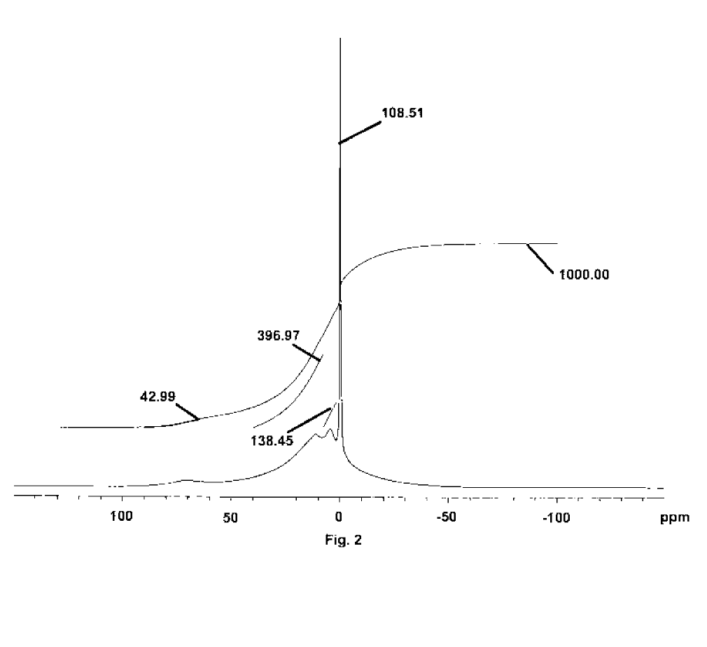

Several double activated aluminum-calcium-glycine solutions were made through the addition of AlCl₃ (32⁰Be) solutions to the Al—Ca-glycine solutions made according to process (B), where the Al—Ca solutions were prepared directly through the reaction of CaO powders with aqueou AlCl₃ solutions and aluminum powders at temperatures of greater than 80° C., followed by the heat treatment with glycine and small amount of water. All the solutions have about 30% anhydrous solids with Al/Cl ratios of 1.0, and 1.1 at different glycine and calcium contents. The results are summarized in Table IV. ²⁷Al NMR of solution 3 are demonstrated in FIG. 2.

TABLE IV

| Experiment | Al/ | | | % | % Band | |
|---|---|---|---|---|---|---|
| Number | Cl | % Ca | % Glycine | Band III | IV | Stability |
| 9 | 1.0 | 1.6 | 3.5 | 53.4 | 26.7 | s.w.^{a}/2 wk |
| 10 | 1.0 | 1.6 | 2.6 | 52.9 | 31.2 | v.s.w.^{b}/2 wk |
| 11 | 1.0 | 1.6 | 1.7 | 38.5 | 26.8 | clear/2 mo |
| 12 | 1.1 | 0.9 | 3.5 | 45.9 | 21.9 | clear/1 mo |
| 13 | 1.1 | 0.9 | 2.6 | 38.9 | 23.0 | clear/1 mo |

^{a}small amount of white precipitate
^{b}very small amount of white precipitate

The data indicate that the double activated Al—Ca-Glycine solutions prepared according process (B) are stable at lower glycine and calcium levels and higher Al/Cl ratio.

### EXAMPLE 5

### Aluminum-Calcium-Betaine Solutions Prepared through Process (B)

Double activated Aluminum-Calcium-Betaine solutions were prepared by mixing aqueous AlCl₃ (32⁰Be) with Al—Ca-Betaine solutions prepared according to process (B), where calcium is from calcium oxide. Table V listed the results of the solutions prepared by this process. All the solutions have about 30% anhydrous solids.

TABLE V

| Experiment | Al/ | % | | | % | |
|---|---|---|---|---|---|---|
| Number | Cl | Ca | % Glycine | % Band III | Band IV | Stability |
| 14 | 1.0 | 1.6 | 5.2 | 53.4 | 24.4 | clear/2 mo |
| 15 | 1.0 | 0.9 | 4.0 | 44.9 | 31.5 | clear |
| 16 | 1.1 | 1.0 | 4.3 | 45.4 | 21.6 | clear |

The Aluminum-Calcium-Betaine solutions are much more stable than the corresponding Aluminum-Calcium-Glycine solutions, which further demonstrates the stabilization effect of betaine.

It will be understood that the present invention is susceptible to numerous changes and modifications as apparent to those skilled in the act. Accordingly, the present invention may be embodied in other specific forms without departing from the spirit of essential attributes of the invention disclosed herein and reference should be made to the appended claims, in the light of the foregoing specifications, as indicating the scope of the invention.

What is claimed is:

1. An aqueous antiperspirant active solution comprising the reaction product of:
(a) about 20% to about 40% anhydrous solid of a basic aluminum chloride (BAC), having the empirical formula
Al₂(OH)₆₋ₐClₐ
where a is a number from about 1.4 to about 2.0;
(b) a buffer selected from the group consisting of betaine (trimethylglycine) and its derivatives and an amino acid; and
(c) a metal ion selected from calcium, strontium and mixtures thereof in amounts of from about of 0.2% to 6% by weight;
having a SEC-HPLC Band III to Band II area ratio of at least 0.7, having a SEC-HPLC Band III plus Band IV of at least 60% of the total area and having a SEC-HPLC Band I content of no more than 2%.

2. The antiperspirant active solution of claim 1 wherein the buffer is betaine.

3. The antiperspirant active solution of claim 1 wherein the buffer is glycine.

4. The antiperspirant active solution of claim 1 wherein the buffer is a mixture of betaine and glycine.

5. The antiperspirant active solution of claim 1 wherein at least 20% of the total SEC-HPLC Band content is Band IV.

6. The antiperspirant active solution of claim 1 wherein at least 60% of the total SEC-HPLC Band content is contained within Band III and Band IV.

7. The antiperspirant active solution of claim 2 wherein betaine is present in amounts of from about 1% to about 8% by weight.

8. The antiperspirant active solution of claim 3 wherein the glycine is present in amounts of from about 1% to about 6% by weight.

9. The antiperspirant active solution of claim 1 wherein a calcium is present in amounts of from about 0.5% to 3% by weight.

10. The antiperspirant active solution of claim 1 wherein the calcium is selected from the group consisting of calcium chloride, calcium nitrate, calcium bromide, calcium citrate, calcium formate, calcium acetate, calcium ascorbate, calcium lactate, calcium carbonate, calcium sulfate, calcium hydroxide, calcium oxide and the mixture thereof.

11. The antiperspirant active solution of claim 1 wherein a strontium is present in amounts of from about 1% to 4% by weight.

12. The antiperspirant active solution of claim 1 wherein the metal ion is strontium and is selected from the group consisting of strontium chloride, strontium nitrate, strontium bromide, strontium citrate, strontium formate, strontium acetate, strontium ascorbate, strontium lactate, strontium carbonate, strontium sulfate, strontium hydroxide, strontium oxide and the mixture thereof.

13. An antiperspirant formulation containing the antiperspirant active of claim 1.

14. An antiperspirant powder obtained by spray drying the solution of claim 1.

15. A method of making the antiperspirant active solution of claim 1 by a double activation effect of a strong acid and heat comprising mixing an acid of the formula AlX₃, wherein X is Cl⁻, Br⁻, I⁻ or NO₃⁻ with a reaction product formed by heating
(i) a water soluble salt selected from calcium, strontium and mixtures thereof;
(ii) a buffering compound selected from betaine and its derivatives; and
(iii) a basic aluminum salt of the empirical formula
Al₂(OH)₆₋ₐXₐ
wherein X is selected from Cl⁻, Br⁻, I⁻, or NO₃⁻, a is from about 1.0 to 1.5, at a temperature of from 40° C. to about reflux until an HPLC Band III/II of at least 0.7 is attained.

16. The method of claim 15 wherein the acid is AlCl₃.

17. The method of claim 15 wherein the acid is a strong inorganic acid.

18. The method of claim 15 wherein the acid is aluminum dichlorohydrate.

19. The method of claim 15 wherein the aluminum to chloride atomic ratio of the resulting solution is from 1.0 to 1.5.

20. The method of claim 15 wherein the aluminum to chloride atomic ratio of the resulting solution is from 1.2 to 1.3.

21. The method of claim 15 wherein the buffering compound is betaine and the salt is a calcium salt.

22. A method of making an antiperspirant active solution by the double activation effect of a strong acid and heat comprising reacting an acid of AlX₃, wherein X is Cl⁻, Br⁻, I⁻ or NO₃⁻ with a reaction product of:
(1) reacting an acidic agent selected from aluminum chloride and hydrochloric acid with a co-reactant base selected from an oxide or hydroxide of calcium and/or strontium;
(2) adding aluminum powder to the reaction product of step (1) and reacting the mixture at temperature in excess of 80° C.; and
(3) adding a buffering agent selected from betaine and its derivatives and an amino acid and mixtures thereof.

23. The method of claim 22 wherein the acid is AlCl₃.

24. The method of claim 22 wherein the acid is a strong inorganic acid.

25. The method of claim 22 wherein the acid is aluminum dichlorohydrate.

26. The method of claim 22 wherein the aluminum to chloride atomic ratio of the resulting solution is from 0.9 to 1.3.

27. The method of claim 22 wherein the aluminum to chloride atomic ratio of the resulting solution is from 1.0 to 1.2.

28. The method of claim 22 wherein the buffering agent is betaine.

29. The method of claim 22 wherein the co-reactant is a calcium salt and the buffering agent is betaine.

30. The method of claim 22 wherein the buffering agent is a mixture of betaine and glycine.

31. The method of claim 22 wherein the co-reactant is a calcium salt and the buffering agent is glycine.
